(19) United States
(12) Patent Application Publication
(10) Pub. No.: US 2017/0224519 A1
(43) Pub. Date: Aug. 10, 2017

(54) KNEE BRACE WITH A MECHANISM ENABLING CHANGING OF CUFF SHELLS

(71) Applicant: Townsend Industries, Inc. d/b/a Townsend Design, Bakersfield, CA (US)

(72) Inventor: Steven S. Knecht, Bakersfield, CA (US)

(73) Assignee: Townsend Industries, Inc. d/b/a Townsend Design, Bakersfield, CA (US)

(21) Appl. No.: 15/017,917

(22) Filed: Feb. 8, 2016

(51) Int. Cl. A61F 5/01

(52) U.S. Cl. CPC A61F 5/0125

(57) ABSTRACT

A knee brace with a connection mechanism that enables quick and easy secure attachment and detachment of a plastic cuff shell to struts of the brace without the need for separate fasteners and the tools required for them via the use of a novel snap connector which will connect by a simple plug in motion, and which is configured to firmly hold the connected strut and prevent it from rotating relative to the cuff shell once connected while also enabling a quick and easy tool-free disconnection of the strut for exchanging of one cuff for another. The thigh and tibia shells can be interchange without removing any screws, rivets or traditional connectors providing a modular construction with the ability to mix and match different size thigh and calf shells, making it possible for a customer to order the product in kit form, or as separate components thereby enabling easy customization.

### BACKGROUND OF THE INVENTION

Field of the Invention

The present invention relates to the field of orthopedic knee braces, and in particular, to those have a hinged pair of femoral and tibial struts, the top end of the femoral struts being connected to a semi-rigid plastic shell forming a thigh cuff, and in some cases, the bottom end of the tibial struts also being connected to semi-rigid plastic shell forming a shin cuff.

Description of Related Art

Knee braces of the initially mentioned type are commercially well known, examples of which are the Townsend full shell knee braces sold by the assignee of the present application. The struts of such knee braces are typically connected to the plastic cuff shells by threaded fasteners or rivets and in some cases are molded into the cuff. In the case of using rivets or molded-on shells, changing of the cuff to fit patients having legs of different sizes is not possible, while in the case of threaded fasteners, such cannot be done without the aid of a tool, is not done quickly, and the fasteners might even loosen later on affecting operability of the brace.

### SUMMARY OF THE INVENTION

The present invention is directed to provided a knee brace with a connection mechanism that avoids the shortcomings of prior art manners of attachment, and enables quick and easy secure attachment and detachment of a plastic cuff shell without the need of separate fasteners and the tools required for them.

This object is achieved in accordance with the invention via the use of a novel snap connector which will connect by a simple plug in motion, and which is configured to firmly hold the connected strut and prevent it from rotating relative to the cuff shell once connected. Furthermore, this object is also achieved by the snap connector enable a quick and easy disconnect of the strut.

### BRIEF DESCRIPTION OF THE DRAWINGS

FIG. 1 shows a knee brace of the type to which the shell connection of the present invention can be applied;

FIG. 2 shows the outer side of femoral cuff in the area for attachment of the femoral strut before attachment thereof;

FIG. 3 is a view similar to that of FIG. 2, but showing the femoral strut attached;

FIG. 4 shows the inner side of femoral cuff before attachment of the femoral strut; and

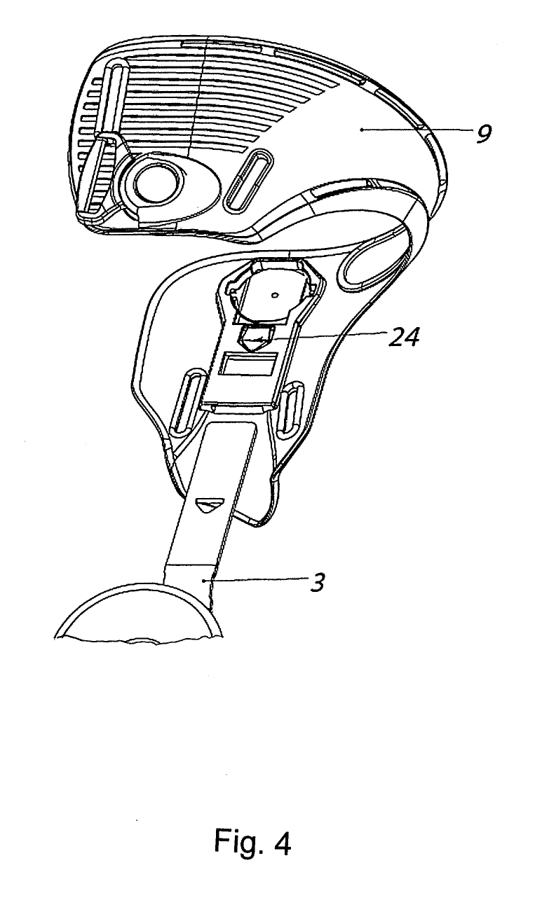

FIG. 5 is a view similar to that of FIG. 4, but showing the outer side of femoral cuff with the femoral strut attached.

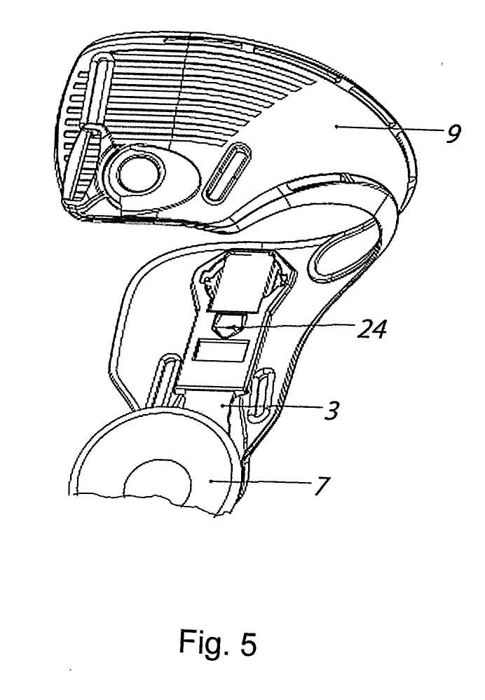

### DETAILED DESCRIPTION OF THE INVENTION

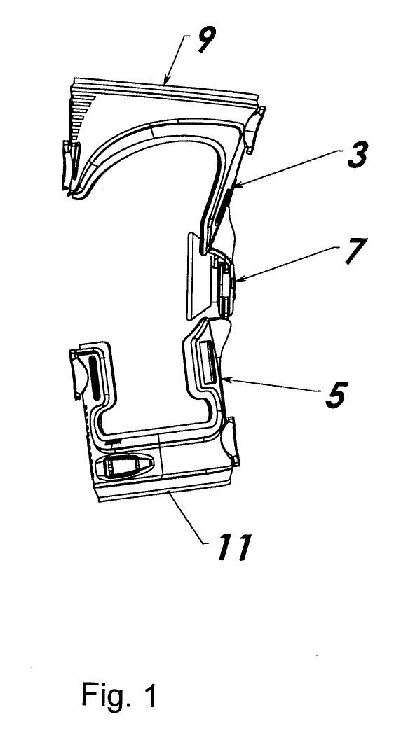

FIG. 1 shows a typical single joint knee brace 1 of the type to which the present invention is intended to be applied. At the lateral side, there is a pair of femoral and tibial struts, 3, 5 which are connected together by a hinge 7; however, the invention is also applicable to dual joint type knee braces in which there is a pair of femoral and tibial struts, 3, 5 which are connected together by a hinge 7 at each side of the knee brace. In the illustrated case, the top end of the femoral strut 3 is connected to a lateral side of a semi-rigid plastic shell 9 that forms a thigh cuff and the bottom end of the tibial struts 5 are also connected to a lateral side of a semi-rigid plastic shell forming a shin cuff shell 11. However, in some cases, the shin cuff shell 11 is replaced by a flexible band. Apart from the quick connect/disconnect mechanism of the invention as described below, the knee brace can otherwise be of virtually any known type having at least one semi-rigid plastic cuff shell including those in which hinged femoral and tibial struts are provided at only one side of the knee brace or at both sides of the knee brace.

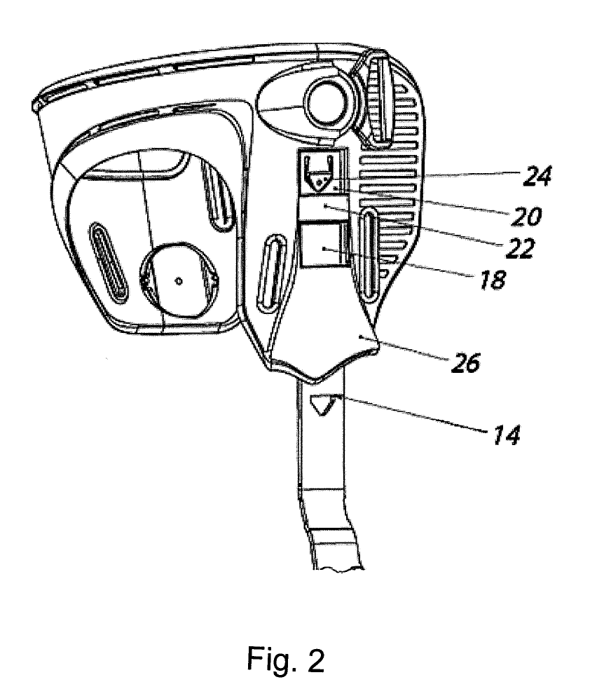

In accordance with the invention, the cuff shell(s) 9, (11) and strut(s) 3, (5) are provided with a quick connect/disconnect mechanism which can be seen most clearly in FIGS. 2 & 3. As shown, each of the struts has a pair of connecting openings, namely, a circular opening 14, and a bar-shaped opening 16. The circular opening 14 is located on the side of the bar-shaped opening 16 that is located facing toward the hinge 7 (i.e., for the femoral struts, the opening 14 is below opening 16, but for the tibial struts it would be above it).

The cuff shell is provided is provided with a pair of strut receiving recesses 18, 20 between which a contact bar 22 is provided. Furthermore, in the recess 20, a cantilevered catch member 24 is formed. The catch member 24 has a pin 24a and a raised bar 24b that are matched in size and position to the openings 14, 16, respectively. A guide surface 26 serves to guide the strut end into the recess 18, under the contact bar 22 and into the recess 20. When the strut is fully inserted into the recess 20, the pin 24a and bar 24b snap into the openings 14, 16, respectively.

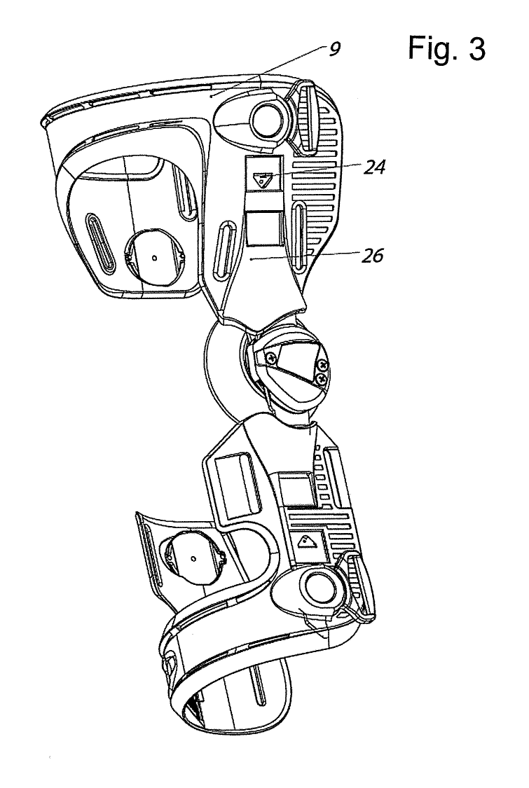

In use of the knee brace, in the position shown in FIGS. 3 & 4, the bar 2b insures that the strut is not able to “wobble” (slide/rotate) in the recesses. The base surfaces of the recesses 18, 20, together with the contact bar 22 hold the strut firmly enough that it cannot rock up and down in the recesses. The cantilevered catch member 24 is connected by an integral hinge to the base of the recess 20 and the length of the pin 24a is long enough that, when the pin 24a is depress far enough for it to be disengaged from the circular opening 14, it will also move the bar 24b sufficiently out of the opening 16 for it to disengage upon pulling of the strut so as to remove it from the cuff shell.

As should be apparent from the foregoing, the invention enables a brace to be provided with a connection mechanism that facilitates quick and easy exchanging of a cuff shell of one size with another of a larger, smaller, or differently configured cuff shell, thereby enabling a quick and easy degree of customization during fitting of the knee brace. This ability to interchange the thigh and tibia shells without the removal of any screws, rivets or traditional connectors provides a modular construction having the ability to mix and match different size thigh and calf shells, making it possible for a customer to order the product in kit form, or as separate components, and essentially build an individual, patient specific brace for a patient who, e.g., does not have a proportionally sized leg.

1-11. (canceled).

12. A knee brace, comprising:
at least one of medial and lateral side femoral struts,
at least one of medial and lateral side tibial struts,
a hinge mechanism connecting said at least one of the medial and lateral side femoral struts to said at least one of the medial and lateral side tibial struts,
a semi-rigid thigh cuff shell,
and a connector mechanism for enabling attachment of said at least one of the medial and lateral side femoral struts to a respective side of the thigh cuff shell, said connector mechanism for producing secure connection and disconnection of the respective femoral strut to the thigh cuff shell wherein the connector mechanism comprises a cantilevered male snap lock member on one of the cuff and strut, the male snap lock member having a projection for snapping into an opening in the other of the strut and cuff when the projection is brought into alignment with the opening, the male snap lock member being deflectable out of said opening for disengaging the catch mechanism.

13. The knee brace according to claim 12, further comprising:
a semi-rigid shin cuff shell, and
a second connector mechanism for enabling attachment of said at least one of the medial and lateral side tibial struts to a respective side of the shin cuff shell, said second connector mechanism also comprising a cantilevered male snap lock member on one of the shin cuff shell and strut, the male snap lock member having a projection for snapping into an opening in the other of the strut and shin cuff shell when the projection is brought into alignment with the opening, the male snap lock member being deflectable out of said opening for disengaging the catch mechanism.

14. The knee brace according to claim 12, wherein the cantilevered male snap lock member is located in a first recess, and wherein a contact bar is located between the first recess and a second recess, the end of the respective strut being insertable into the recesses with the bar located on an opposite side thereof from a base surface of the recesses.

15. The knee brace according to claim 14, further comprising a guide surface shaped so as to guide the end of the respective strut into the recesses and under the contact bar.

16. A knee brace kit, comprising the following components packaged in an unassembled condition:
at least one of medial and lateral side femoral struts,
at least one of medial and lateral side tibial struts,
a hinge mechanism for use in connecting said at least one of the medial and lateral side femoral struts to said at least one of the medial and lateral side tibial struts,
a semi-rigid thigh cuff shell,
and a connector mechanism for enabling attachment of said at least one of the medial and lateral side femoral struts to a respective side of the thigh cuff shell, wherein the connector mechanism comprises a cantilevered male snap lock member on one of the cuff and strut, the male snap lock member having a projection for snapping into an opening in the other of the strut and cuff when the projection is brought into alignment with the opening, the male snap lock member being deflectable out of said opening for disengaging the catch mechanism.

17. A knee brace, comprising:
at least one of medial and lateral side femoral struts,
at least one of medial and lateral side tibial struts,
a hinge mechanism connecting said at least one of the medial and lateral side femoral struts to said at least one of the medial and lateral side tibial struts,
a semi-rigid thigh cuff shell,
and a connector mechanism for enabling attachment of said at least one of the medial and lateral side femoral struts to a respective side of the thigh cuff shell, said connector mechanism comprising a catch mechanism for producing secure connection and disconnection of the respective femoral strut to the thigh cuff shell,
further comprising:
a semi-rigid shin cuff shell, and
a second connector mechanism for enabling attachment of said at least one of the medial and lateral side tibial struts to a respective side of the shin cuff shell, said connector mechanism comprising a catch mechanism for producing secure connection and disconnection of the respective tibial strut to the shin cuff shell,
wherein said connector mechanism comprises a cantilevered catch on the cuff having projections for engaging like-shaped openings in the struts, and
wherein the cantilevered catch is located in a first recess, and wherein a contact bar is located between the first recess and a second recess, the end of the respective strut being insertable into the recesses with the bar located on an opposite side thereof from a base surface of the recesses.
